(19) United States
(12) Patent Application Publication
(10) Pub. No.: US 2010/0062986 A1
(43) Pub. Date: Mar. 11, 2010

(54) METHOD TO TREAT BLOOD CELL DEPLETION

(75) Inventor: Hal Siegel, Paradise Valley, AZ (US)

Correspondence Address: JONES DAY, 222 EAST 41ST ST, NEW YORK, NY, 10017, US

(73) Assignee: ImmuneRegen Biosciences, Inc., Scottsdale, AZ (US)

(21) Appl. No.: 12/301,426

(22) PCT Filed: May 30, 2007
(86) PCT No.: PCT/US07/12661

§ 371 Date: Nov. 23, 2009

Related U.S. Application Data
(60) Provisional application No. 60/809,391, filed on May 31, 2006.

(51) Int. Cl. A61K 38/08; A61P 7/00

(52) U.S. Cl. 514/15

(57) ABSTRACT

Neuropeptides are used to treat mammals which have depletion of count of one or more blood cell types. Intramuscular administration or administration via inhalation causes the blood cell count to increase.

### FIELD OF THE INVENTION

The invention relates to the field of hematology. In particular, it relates to depletion of blood cells, from whatever cause. More particularly it relates to treatment of individuals who have a depleted blood cell population.

### BACKGROUND OF THE INVENTION

Red blood cells express hemoglobin which binds to oxygen and transports it around the body. Proper oxygenation of tissues is dependent on the amount and function of red blood cells in general, and hemoglobin in particular. The normal range of red blood cell counts is 4.7 to 6.1×10⁶ cells/microliter for males and 4.2 to 5.4×10⁶ cells/microliter for females.

Red blood cells may be depleted due to anemia, hemolytic anemia, hemorrhage, bone marrow failure, kidney disease, leukemia, multiple myeloma, malnutrition, genetic deficiency in glucose-6-phosphate dehydrogenase, and deficiency of iron, folate, vitamin B-12, or vitamin B-6. Drugs such as chloramphenicol, hydantoins, and quinidine can decrease the number of red blood cells.

Platelets are required for proper clotting of blood. Low platelet counts may result from bone marrow malfunction (due to, e.g., aplastic anemia, cancer, infections), immune thrombocytopenic purpura, drug induced thrombocytopenia (such as cancer chemotherapy), thrombotic thrombocytopenic purpura, primary thrombocythemia, disseminated intravascular coagulation, hypersplenism, massive blood transfusion, leukemia, hemolytic anemia, and spleen or liver disorders. Normal values for platelets are 15,000 to 400,000/mm³.

Leukocytes are required for defense against infectious agents, such as bacteria, fungi, parasites, and viruses. Leukocytes include neutrophils, band cells, T lymphocytes, B lymphocytes, monocytes, eosinophils, and basophils. Low numbers of leukocytes may be caused by bone marrow failure, toxic substances, autoimmune disease, spleen or liver disorders, and radiation exposure.

Hematocrit is the percentage of red blood cells in whole blood. It is a measure of the number and size of erythrocytes. Normal values of hematocrit for males is 40.7 to 50.3% and for females 36.1 to 44.3%. Normal values of hemoglobin are 13.8 to 17.2 gm/dL for male humans and 12.1 to 15.1 gm/dL for female humans. Lower than normal hemoglobin may be caused by anemia, hemolytic anemia, erythropoietin deficiency, transfusion reaction, bleeding, lead poisoning, malnutrition, overhydration.

There is a continuing need in the art for tools to help increase counts of blood cells in situations where they are depleted.

### BRIEF SUMMARY OF THE INVENTION

According to the present invention a method is provided for treating depletion of one or more blood cell types. An effective amount of Substance P or an analog thereof is injected intramuscularly to a mammal in which one or more blood cell types is depleted. The analog is selected from the group consisting of [Met-OH¹¹]-substance P, [Met-OMe¹¹]-substance P, [Nle¹¹]-substance P, [Pro⁹]-substance P, [Sar⁹]-substance P, [Tyr⁸]-substance P, [p-Cl-Phe^{7,8}]-substance P, [Sar⁹, Met(0₂)¹¹]-substance P, and analogs having the amino acid backbone RPKPQQFFGLM-NH₂ (SEQ ID NO: 1). The count of at least one of the blood cell types, such as erythrocytes, leukocytes, and platelets, increases as a result of the injection.

According to the present invention another method is provided for treating depletion of one or more blood cell types. An effective amount of Substance P or an analog thereof is administered via inhalation to a mammal in which one or more blood cell types is depleted. The analog is selected from the group consisting of [Met-OH¹¹]-substance P, [Met-OMe¹¹]-substance P, [Nle¹¹]-substance P, [Pro⁹]-substance P, [Sar⁹]-substance P, [Tyr⁸]-substance P, [p-Cl-Phe^{7,8}]-substance P, [Sar⁹, Met(0₂)¹¹]-substance P, and analogs having the amino acid backbone RPKPQQFFGLM-NH₂ (SEQ ID NO: 1). The count of at least one of the blood cell types, such as erythrocytes, leukocytes, and platelets, increases as a result of the administration.

### DETAILED DESCRIPTION OF THE INVENTION

It is a discovery of the present invention that substance P or its bioactive analogs can reverse blood cell depletion. The depleted blood cell population may be one or more of the following: erythrocytes, leukocytes, and platelets. Mammals, and in particular humans, who have depletions of one or more blood cell types, i.e., counts that are below the normal range, can be treated, e.g., via intramuscular injection, to reverse the depletion and to increase the count of one or more blood cell types. Preferably the blood cell types return to the normal range upon treatment.

Substance P (RPKPQQFFGLM-NH₂; SEQ ID NO: 1) is synthesized as a glycine-extended precursor and converted post-translationally to the biologically active, C-terminal amide. Substance P. or a bioactive analog thereof such as Sar⁹, Met(O₂)¹¹-Substance P can be administered to treat individuals who have depleted blood cell population(s). The bioactive analog can be selected from the group consisting of [Met-OH¹¹]-substance P, [Met-OMe¹¹]-substance P, [Nle¹¹]-substance P, [Pro⁹]-substance P, [Sar⁹]-substance P, [Tyr⁸]-substance P, Sar⁹, Met(O₂)¹¹-Substance P, and [p-Cl-Phe^{7,8}]-substance P. Other compounds which function in the same way can be identified by their ability to compete with substance P for binding to its receptor (NK-1) or for their ability to agonize the NK-1 receptor. Compounds which have the same amino acid backbone as substance P can be routinely modified and tested for receptor agonist activity. Routine assays for such activities are known in the art and can be used.

The substance P or analog can be administered by any method known in the art, including via intramuscular injection. Formulations using mechanisms to ensure targeting or modifications in availability such as liposome preparations, conjugates with targeting molecules (e.g., antibodies, lectins), and incorporation into stents, implants or other physical delivery vehicles can also be used. Intravenous, aerosol inhalation, topical, intratracheal, intrabronchial, intranasal, subcutaneous, sublingual, and oral administrations can also be used. Suitable concentration ranges of substance P or its bioactive analog in an aerosol administered is between 1 μM and 5000 μM, including 50 μM to 500 μM, 1-10 μM, 50-100 μM, 500-1000 μM, and 1000-5000 μM. Exemplary concentrations which can be used include 1, 10, 50, 75, 100, 300 and 1000 μM. For intramuscular injections, a volume of about 0.1 to 1.0 ml/kg of body weight can be used.

The methods of the present invention can be applied to any mammal, including humans, horses, sheep, primates such as monkeys, apes, gibbons, chimpanzees, rodents such as mice, rats, guinea pigs, hamsters, ungulates such as cows, and camelids, such as camels and llamas. Depletion of blood cells can be caused by multiple inciting agents: hematological diseases, radiation exposure (whether therapeutic or weaponized or accidental), side effects of drug usage, etc. Inciting causes may be anemia, autoimmune disease, bleeding, bone marrow failure, bone marrow malfunction (due to, e.g., aplastic anemia, cancer, infections), deficiency of iron, folate, vitamin B-12, or vitamin B-6, drug induced thrombocytopenia (such as cancer chemotherapy), disseminated intravascular coagulation, erythropoietin deficiency, genetic deficiency in glucose-6-phosphate dehydrogenase, hemolytic anemia, hemorrhage, hypersplenism, immune thrombocytopenic purpura, kidney disease, lead poisoning, leukemia, malnutrition, massive blood transfusion, multiple myeloma, overhydration, primary thrombocythemia, radiation exposure, spleen or liver disorders, thrombotic thrombocytopenic purpura, toxic substances, and transfusion reaction.

The methods of the present invention can be used to increase the numbers of any blood cell class or subclass. These include erythrocytes, leukocytes, and platelets. It further includes neutrophils, band cells, T lymphocytes, B lymphocytes, monocytes, eosinophils, and basophils. More particularly, these include T helper cells, CD45 positive cells, antigen presenting cells, CD4 positive cells, CD8 positive cells, and T cells expressing Toll Like Receptor (TLR) types.

Determining counts of blood cell types, classes, subclasses and subtypes can be done according to any method known in the art. Many such methods are automated and are provided in a standard complete blood count. Parameters of the blood which can be determined and used for monitoring include red blood cell count, white blood cell count, platelet count, hemoglobin concentration, hematocrit, MCV, red blood cell size, count of any blood cell type or subtype, etc. The determining of counts or other blood parameters can be performed before and/or after treatment and can be performed multiple times for continued monitoring.

While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

### Example

One purpose of the study is to determine the effects of Radilex™ ([Sar⁹, Met(0₂)¹¹]-Substance P) after exposure to a lethal dose of ⁶⁰Co radiation exposure. Another purpose of the study is to determine if intra-muscular injections of Radilex™ can be as effective as aerosol inhalation of Radilex™ at 8Gy ⁶⁰Co radiation level.

Blood draws (2 animals per group rotating every 5 days) for erythrocytes, leukocytes and platelets were taken to assess anemia, neutropenia and thrombocytopenia. Counts of erythrocytes, leukocytes and platelets were taken to assess anemia, neutropenia and thrombocytopenia and act as indicators of bone marrow damage and/or immune system destruction.

The mice received Radilex™ treatment within 2 hours of radiation exposure. The Radilex™ dosage was administered in a volume of 0.01 ml in a daily single treatment via IM injection into the hind limb muscle a single time, for the duration of the study (30 days), or until death.

The following regimens were tested on groups of 9-11 mice:

10 mM Radilex™ single intramuscular treatment

1 mM Radilex™ single intramuscular treatment

1 mM Radilex™ daily intramuscular treatment

75 uM Radilex™ single intramuscular treatment

75 uM Radilex™ daily intramuscular treatment

10 uM Radilex™ daily via nebulizer

50 uM Radilex™ daily via nebulizer

75 uM Radilex™ daily via nebulizer

100 uM Radilex™ daily via nebulizer

200 uM Radilex™ single administration via nebulizer at 2 hours post radiation exposure

1 mM Radilex™ single administration via nebulizer at 2 hours post radiation exposure

10 mM Radilex™ single administration via nebulizer at 2 hours post radiation exposure

Confirmation of Radioprotection. Effects of Lethal Exposure Reversed by Radilex™.

Neutropenia and Anemia Reversed in Surviving Animals.

The normal white cell count for C57BL/6J is 6.2±2.7 K/microliter for males, 5.9±1.1 for females. Normal hematocrit is 45.4±1.7% for males, 46.2±1.2% for females. The mice that did not survive our highest radiation dose, 8 Gy*, had white cell counts 1/10 of normal or less, and hematocrits of less than half normal. Mice that did survive had, on average, white cell counts at 1/10 of normal or higher, and hematocrits at half normal or higher. It is clear, although we cannot prove it statistically since our sample size per group is too small, that more mice survived the 8 Gy dose than should have survived, suggesting efficacy of drug treatment.

1. A method for treating depletion of one or more blood cell types, comprising: injecting intramuscularly an effective amount of Substance P or an analog thereof to a mammal in which one or more blood cell types is depleted, wherein said analog is selected from the group consisting of [Met-OH¹¹]-substance P, [Met-OMe¹¹]-substance P, [Nle¹¹]-substance P, [Pro⁹]-substance P, [Sar⁹]-substance P, [Tyr⁸]-substance P, [p-Cl-Phe^{7,8}]-substance P, and [Sar⁹, Met(O₂)¹¹]-substance P, wherein the blood cell types are selected from the group consisting of erythrocytes, leukocytes, and platelets, whereby the count of at least one of the blood cell types increases.

2. The method of claim 1 wherein Substance P is injected.

3. The method of claim 1 wherein [Met-OH¹¹]-Substance P is injected.

4. The method of claim 1 wherein [Met-OMe¹¹]-Substance P is injected.

5. The method of claim 1 wherein [Nle¹¹]-Substance P is injected.

6. The method of claim 1 wherein [Pro⁹]-Substance P is injected.

7. The method of claim 1 wherein [Sar⁹]-Substance P is injected.

8. The method of claim 1 wherein [Tyr⁸]-Substance P is injected.

9. The method of claim 1 wherein [p-Cl-Phe^{7,8}]-Substance P is injected.

10. The method of claim 1 wherein [Sar⁹, Met(O₂)¹¹]-Substance P is injected.

11. The method of claim 1 wherein the mammal is a human.

12. The method of claim 11 wherein the human has a leukocyte count of less than 2000 prior to said step of injecting.

13. The method of claim 11 wherein the human has a leukocyte count of less than 1500 prior to said step of injecting.

14. The method of claim 11 wherein the human has a leukocyte count of less than 1000 prior to said step of injecting.

15. The method of claim 11 wherein the human has a leukocyte count of less than 500 prior to said step of injecting.

16. The method of claim 11 wherein the human has an erythrocyte count per microliter prior to said step of injecting of less than 4.2×10⁶ if a female and less than 4.7×10⁶ if a male.

17. The method of claim 11 wherein prior to said step of injecting. the hemoglobin count of the human is less than 12 grams per deciliter if a female or less than 13.8 grams per deciliter if a male.

18. The method of claim 11 wherein the hematocrit of the human prior to said step of injecting. is less than 40% of a male and less than 36% if a female.

19. The method of claim 11 wherein the platelet count of the human is less than 150,000 per square millimeter prior to said step of injecting.

20. The method of claim 1 further comprising monitoring one or more blood cell type counts of the mammal after said step of injecting.

21. The method of claim 1 further comprising monitoring one or more blood cell type counts of the mammal prior to said step of injecting.

22. The method of claim 1 wherein after the step of injecting the count of at least one of the blood cell types is increased to a value in the normal range for the mammal.

23-25. (canceled)

26. The method of claim 1 wherein the mammal was exposed to radiation prior to said step of injecting.

27. The method of claim 1 wherein the mammal was exposed to a lethal dose of radiation prior to said step of injecting.

28. The method of claim 1 wherein the blood cell type count of the mammal has been reduced due to disease prior to said step of injecting.

29. The method of claim 1 wherein the blood cell type count of the mammal has been is reduced due to a side effect of a drug prior to said step of injecting.

30. The method of claim 1 wherein at least one blood cell type count of the mammal is determined before and after said step of injecting.

31. A method for treating depletion of one or more blood cell types, comprising: administering an effective amount of Substance P or an analog thereof via inhalation to a mammal in which one or more blood cell types is depleted, wherein said analog is selected from the group consisting of [Met-OH¹¹]-substance P, [Met-OMe¹¹]-substance P, [Nle¹¹]-substance P, [Pro⁹]-substance P, [Sar⁹]-substance P, [Tyr⁸]-substance P, [p-Cl-Phe^{7,8}]-substance P, and [Sar⁹, Met(O₂)¹¹]-substance P, wherein the blood cell types are selected from the group consisting of erythrocytes, leukocytes, and platelets, whereby the count of at least one of the blood cell types increases.

32-60. (canceled)


SEQUENCE LISTING (1 sequence)

SEQ ID NO: 1 - protein, 11 aa, Homo sapiens
RPKPQQFFGLM